(19) United States
(12) Patent Application Publication
(10) Pub. No.: US 2009/0203589 A1
(43) Pub. Date: Aug. 13, 2009

(54) CHEMICALLY MODIFIED HUMAN GROWTH HORMONE RECEPTOR ANTAGONIST CONJUGATES

(75) Inventors: Thomas J. Girard, Chesterfield, MO (US); Rory F. Finn, Chesterfield, MO (US); Ned R. Siegel, Chesterfield, MO (US)

Correspondence Address: PFIZER INC.;PATENT DEPARTMENT, Bld 114 M/S 114, EASTERN POINT ROAD, GROTON, CT, 06340, US

(73) Assignee: PFIZER INC.

(21) Appl. No.: 11/815,842

(22) PCT Filed: Jan. 31, 2005
(86) PCT No.: PCT/IB2005/000228

§ 371 Date: Dec. 11, 2008

Related U.S. Application Data
(60) Provisional application No. 60/543,078, filed on Feb. 9, 2004.

(51) Int. Cl. A61K 38/16; C07K 17/00; A61P 3/10; A61P 35/00; A61P 3/00

(52) U.S. Cl. 514/12; 530/409

(57) ABSTRACT

The present invention provides a chemically modified human Growth Hormone (hGH) receptor antagonists prepared by attaching a single polyethylene glycol moiety to the N-terminus. The chemically-modified protein according to the present invention have decreased PEGylation heterogeneity and which may also have increased binding affinity.

The present application claims priority to U.S. application No. 60/543,078 filed Feb. 9, 2004, which is incorporated by reference in its entirety as if written herein.

### FIELD OF THE INVENTION

The present invention relates to a chemical modification of a human Growth Hormone Receptor Antagonist by which the chemical and/or physiological properties of Growth Hormone Receptor antagonist can be changed. The modified Growth Hormone Receptor antagonist have decreased PEGylation heterogeneity and may also have decreased plasma residency duration, decreased clearance rate, improved stability, decreased antigenicity, increased binding affinity, increased potency or a combination thereof. The present invention also relates to processes for the generation and modification of Growth Hormone Receptor antagonist. In addition, the present invention relates to pharmaceutical compositions comprising the modified Growth Hormone Receptor antagonist. A further embodiment is the use of the modified Growth Hormone Receptor antagonist for the treatment of growth and development disorders.

### BACKGROUND OF THE INVENTION

Human growth hormone (hGH) is a protein comprising a single chain of 191 amino acids cross-linked by two disulphide bridges and the monomeric form has a molecular weight of 22 kDa.

It has previously been shown that monovalent phage display (Bass et al., Proteins, 8: 309-314 [1990]) can be used to improve the affinity of Site 1 in hGH for the hGHbp. Lowman et al., Biochemistry, 30: 10832-10838 (1991). Modest improvements in binding affinity (3 to 8-fold tighter than wild-type hGH) were produced by sorting three independent libraries each mutated at four different codons in Site 1. An hGH mutant slightly enhanced in binding affinity for Site 1 and blocked in its ability to bind Site 2 was a better antagonist of the hGH receptor than the Site 2 mutant alone. Fuh et al., Science, 256: 1677-1680 (1992).

It has been disclosed that the lysine residues of hGH and bGH are involved in the interaction of hGH and bGH with somatotropic receptors, with the structure-function relationship particularly implicating the lysine or arginine residues at positions 41, 64, 70, and 115. Martal et al., FEBS Lett., 180: 295-299 (1985). Lysine residues were chemically modified by methylation, ethylation, guanidination, and acetimidination, resulting in reduced activity by radioreceptor assay.

Additional improvements in Site 1 affinity were obtained by mutating more residues per library to obtain an even better antagonist that can have utility in treating conditions of GH excess such as acromegaly. Modifications of Site II can generate antagonists, however, these molecules are of limited utility due to their short circulating half-lives.

It is generally observed that physiologically active proteins administered into a body can show their pharmacological activity only for a short period of time due to their high clearance rate in the body. Furthermore, the relative hydrophobicity of these proteins may limit their stability and/or solubility.

For the purpose of decreasing the clearance rate, improving stability or abolishing antigenicity of therapeutic proteins, some methods have been proposed wherein the proteins are chemically modified with water-soluble polymers. Chemical modification of this type may block effectively a proteolytic enzyme from physical contact with the protein backbone itself, thus preventing degradation. Chemical attachment of certain water-soluble polymers may effectively reduce renal clearance due to increased hydrodynamic volume of the molecule. Additional advantages include, under certain circumstances, increasing the stability and circulation time of the therapeutic protein, increasing solubility, and decreasing immunogenicity. Poly(alkylene oxide), notably poly(ethylene glycol) (PEG), is one such chemical moiety that has been used in the preparation of therapeutic protein products (the verb “pegylate” meaning to attach at least one PEG molecule). The attachment of poly(ethylene glycol) has been shown to protect against proteolysis, Sada, et al., J. Fermentation Bioengineering 71: 137-139 (1991), and methods for attachment of certain poly(ethylene glycol) moieties are available. See U.S. Pat. No. 4,179,337, Davis et al., “Non-Immunogenic Polypeptides,” issued Dec. 18, 1979; and U.S. Pat. No. 4,002,531, Royer, “Modifying enzymes with Polyethylene Glycol and Product Produced Thereby,” issued Jan. 11, 1977. For a review, see Abuchowski et al., in Enzymes as Drugs. (J. S. Holcerberg and J. Roberts, eds. pp. 367-383 (1981)).

Other water-soluble polymers have been used, such as copolymers of ethylene glycol/propylene glycol, carboxymethylcellulose, dextran, poly(vinyl alcohol), poly(vinyl pyrrolidone), poly(-1,3-dioxolane), poly(-1,3,6-trioxane), ethylene/maleic anhydride copolymer, poly-amino acids (either homopolymers or random copolymers).

A number of examples of pegylated therapeutic proteins have been described. ADAGEN®, a pegylated formulation of adenosine deaminase, is approved for treating severe combined immunodeficiency disease. ONCASPAR®, a pegylated L-asparaginase has been approved for treating hypersensitive ALL patients. Pegylated superoxide dismutase has been in clinical trials for treating head injury. Pegylated α-interferon (U.S. Pat. Nos. 5,738,846, 5,382,657) has been approved for treating hepatitis; pegylated glucocerebrosidase and pegylated hemoglobin are reported to have been in preclinical testing. Another example is pegylated IL-6, EF 0 442 724, entitled, “Modified hIL-6,” which discloses poly(ethylene glycol) molecules added to IL-6.

Another specific therapeutic protein, which has been chemically modified, is granulocyte colony stimulating factor, (G-CSF). G-CSF induces the rapid proliferation and release of neutrophilic granulocytes to the blood stream, and thereby provides therapeutic effect in fighting infection. European patent publication EP 0 401 384, published Dec. 12, 1990, entitled, “Chemically Modified Granulocyte Colony Stimulating Factor,” describes materials and methods for preparing G-CSF to which poly(ethylene glycol) molecules are attached. Modified G-CSF and analogs thereof are also reported in EP 0 473 268, published Mar. 4, 1992, entitled “Continuous Release Pharmaceutical Compositions Comprising a Polypeptide Covalently Conjugated To A Water Soluble Polymer,” stating the use of various G-CSF and derivatives covalently conjugated to a water soluble particle polymer, such as poly(ethylene glycol). A modified polypeptide having human granulocyte colony stimulating factor activity is reported in EP 0 335 423 published Oct. 4, 1989. Provided in U.S. Pat. No. 5,824,784 are methods for N-terminally modifying proteins or analogs thereof, and resultant compositions, including novel N-terminally chemically modified G-CSF compositions. U.S. Pat. No. 5,824,778 discloses chemically modified G-CSF.

For poly(ethylene glycol), a variety of means have been used to attach the poly(ethylene glycol) molecules to the protein. Generally, poly(ethylene glycol) molecules are connected to the protein via a reactive group found on the protein.

Amino groups, such as those on lysine residues or at the N-terminus, are convenient for such attachment. For example, Royer (U.S. Pat. No. 4,002,531, above) states that reductive alkylation was used for attachment of poly(ethylene glycol) molecules to an enzyme. EP 0 539 167, published Apr. 28, 1993, Wright, “Peg Imidates and Protein Derivatives Thereof” states that peptides and organic compounds with free amino group(s) are modified with an imidate derivative of PEG or related water-soluble organic polymers. Chamow et al., Bioconjugate Chem. 5: 133-140 (1994) report the modification of CD4 immunoadhesin with monomethoxypoly(ethylene glycol) aldehyde via reductive alkylation. The authors report that 50% of the CD4-Ig was MePEG-modified under conditions allowing control over the extent of pegylation. Id. at page 137. The authors also report that the in vitro binding capability of the modified CD4-Ig (to the protein gp 120) decreased at a rate correlated to the extent of MePEGylation Ibid. U.S. Pat. No. 4,904,584, Shaw, issued Feb. 27, 1990, relates to the modification of the number of lysine residues in proteins for the attachment of poly(ethylene glycol) molecules via reactive amine groups.

Many methods of attaching a polymer to a protein involve using a moiety to act as a linking group. Such moieties may, however, be antigenic. A tresyl chloride method involving no linking group is available, but this method may be difficult to use to produce therapeutic products as the use of tresyl chloride may produce toxic by-products. See Francis et al., In: Stability of protein pharmaceuticals: in vivo pathways of degradation and strategies for protein stabilization (Eds. Ahern, T. and Manning, M. C.) Plenum, N.Y., 1991) Also, Delgado et al., “Coupling of PEG to Protein By Activation With Tresyl Chloride, Applications In Immunoaffinity Cell Preparation”, in Separations Using Aqueous Phase Systems, Applications In Cell Biology and Biotechnology, Fisher et al., eds. Plenum Press, New York, N.Y., 1989 pp. 211-213.

See also, Rose et al., Bioconjugate Chemistry 2: 154-159 (1991) which reports the selective attachment of the linker group carbohydrazide to the C-terminal carboxyl group of a protein substrate (insulin).

WO 97/11178 relates to hGH receptor antagonists that have been modified with PEG at multiple sites (2-7) to primary amino groups. One such hGH receptor antagonist, Pegvisomant® contains on average 5 attachments of 5K PEG moieties attached to the human growth hormone receptor antagonist B2036 (B2036 is GH that is modified at eight residues to enhance site I binding and modified at residue 120 to lysine) as described by Olson et al. Poly(ethylene glycol) Chemistry and Biological Applications, Eds., Harris and Zalipsky, 1997.

However, it is still desirable to have a mono-PEGylated human growth hormone receptor antagonist conjugate with decreased PEGylation heterogeneity and which may also have increased receptor affinity. The present invention provides human growth hormone receptor antagonist-PEG conjugates having a single chemical modification at the N-terminus which may also have increased affinity to its receptor and which may also have decreased clearance rate, increased plasma residency duration, improved solubility, increased stability, decreased antigenicity, increased potency or combinations thereof.

### SUMMARY OF THE INVENTION

The present invention relates to chemically modified human growth hormone receptor antagonists having decreased PEGylation heterogeneity which may also have increased binding affinity, and which may have improved chemical or physiological properties selected from but not limited to decreased clearance rate, increased plasma residency duration, increased stability, improved solubility, and decreased antigenicity. Thus, as described below in more detail, the present invention has a number of aspects relating to chemically modifying human growth hormone receptor antagonists as well as specific modifications using a variety of Butyraldehyde poly(ethylene glycol) moieties.

The present invention also relates to methods of producing the chemically modified human growth hormone receptor antagonists.

The present invention also relates to compositions comprising the chemically modified human growth hormone receptor antagonists.

The chemically modified human growth hormone receptor antagonists of the present invention, are useful in treating conditions in which the inhibition of GH action is desirable. Particularly amenable to treatment with chemically modified human growth hormone receptor antagonists are conditions in which a reduction of circulating levels of GH or of a mediator of GH action, such as IGF-I, provides a therapeutic benefit. Such conditions include conditions of GH excess such as, for example, giantism and acromegaly. Giantism results from GH excess before puberty, when the long bone growth is still possible. Acromegaly results from GH excess after puberty, when the long bones have fused. Acromegaly is characterized by bony overgrowth and soft tissue swelling as well as hypertrophy of internal organs, especially the heart. Acromegaly is typically caused by a pituitary tumor that secretes GH. The hallmarks of the disease are high levels of circulating GH and IGF-I. The chemically modified human growth hormone receptor antagonists of the present invention are presently believed to offer a significant therapeutic benefit by inhibiting GH action.

The chemically modified human growth hormone receptor antagonists are also useful in treating the other conditions in which the inhibition of GH action provides therapeutic benefit. Examples include diabetes and its complications, such as for instance diabetic retinopathy and diabetic nephropathy. Diabetic retinopathy is characterized by proliferation of the cells making up the retinal blood vessels, growth of new vessels on top of the retina (neovascularization), development of microaneurysms, and leakage of fluid into the surrounding retinal tissue. The early hallmarks of diabetic nephropathy are renal hypertrophy and hyperfiltration. As the disease progresses, diffuse enlargement of the mesangial cells (which support the filtration apparatus of the kidney) is observed, accompanied by an absolute increase in the number of mesangial cells.

Vascular eye diseases that, like diabetic retinopathy, involve proliferative neovascularization are also believed to be amenable to treatment with antagonist human growth hormone receptor antagonist. Examples include retinopathy of prematurity, retinopathy associated with sickle cell anemia, and age-related macular degeneration, which is the most common cause of vision loss in persons over 55.

Other conditions in which the reduction of GH levels is presently believed to provide a therapeutic benefit include malignancies that respond to GH, or a mediator of GH action (such as IGF-1), by growing (hereinafter “GH-responsive malignancies”). Examples of GH-responsive malignancies include Wilm's tumor, various sarcomas (e.g., osteogenic sarcoma), breast, colon, prostate, and thyroid cancer.

The chemically modified human growth hormone receptor antagonists of the present invention inhibit the growth of cells expressing receptors to which the variants bind. A wide variety of tissues express such receptors. For example, GH receptor mRNA is expressed in cell lines from normal placenta, thymus, brain, salivary gland, prostate, bone marrow, skeletal muscle, trachea, spinal cord, retina, lymph node and from Burkitt's lymphoma, colorectal carcinoma, lung carcinoma, lymphoblastic leukemia, and melanoma. Thus, it is presently believed that chemically modified human growth hormone receptor antagonists of the present invention are generally useful in treating cancers that express receptors to which the variants bind.

### BRIEF DESCRIPTION OF THE DRAWINGS

FIG. 1 is the amino acid sequence (SEQ ID NO:1) of the hGH receptor antagonist B2036.

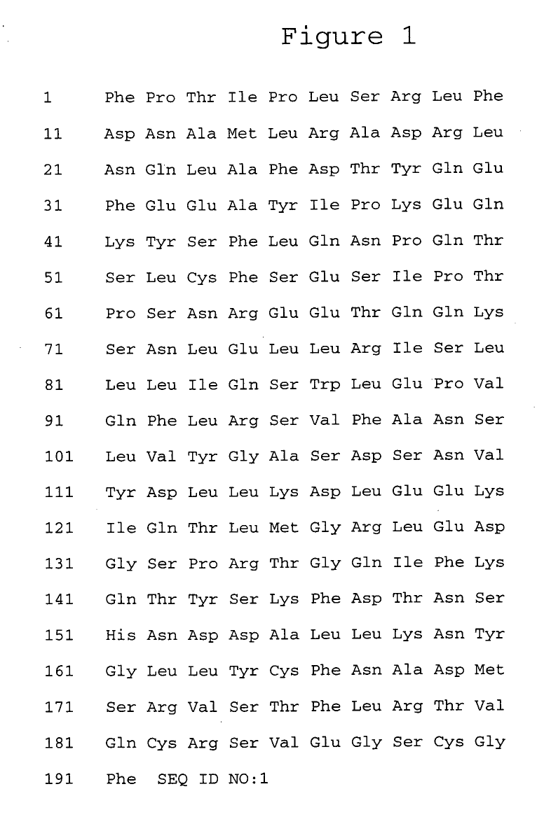

FIG. 2 is a size exclusion chromatogram of branched 40k ALD-B2036 conjugate. Panel a is the chromatogram of the reaction mixture. Peak 2 with a retention time of 19.883 is di-PEGylated product. Peak 4 with a retention time 33.883 is the unreacted B2036 protein. Panel b is the chromatogram of the purified mono-PEGylated product showing a single peak with a retention time of 22.700.

FIG. 3 shows the IGF-1 levels in cynomolgus monkeys following a single subcutaneous dose (0.3 mpk or 1.0 mpk) of N-terminally monopegylated 30K ALD-B2036 conjugate.

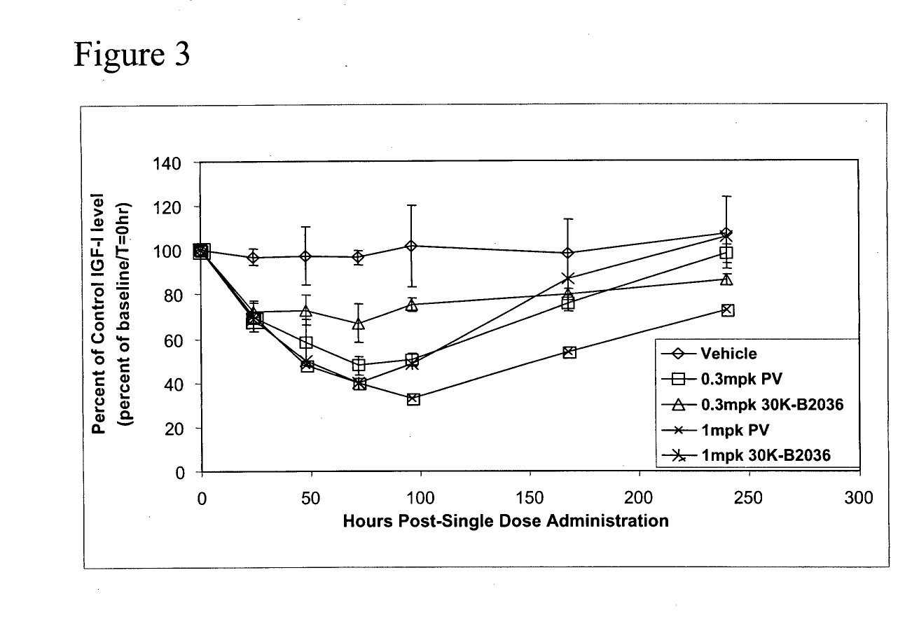

### DETAILED DESCRIPTION

Human growth hormone receptor antagonists are members of a family of recombinant proteins, described in U.S. Pat. No. 5,849,535, U.S. Pat. No. 5,534,617, U.S. Pat. No. 6,143,523, U.S. Pat. No. 6,022,711, U.S. Pat. Nos. 5,834,598, and 5,688,666, which also describe their recombinant production and methods of use.

Any purified and isolated human growth hormone receptor antagonists, which is produced by host cells such as E. coli and animal cells transformed or transfected by using recombinant genetic techniques, may be used in the present invention. Additional human growth hormone receptor antagonists are described in U.S. Pat. No. 4,871,835. Among them, human growth hormone receptor antagonists, which are produced by the transformed E. coli, are particularly preferable. Such human growth hormone receptor antagonists may be obtained in large quantities with high purity and homogeneity. For example, the above human growth hormone receptor antagonists may be prepared according to a method disclosed in U.S. Pat. Nos. 4,342,832, 4,601,980; U.S. Pat. No. 4,898,830; U.S. Pat. No. 5,424,199; U.S. Pat. Nos. 5,795,745 5,849,535, U.S. Pat. No. 5,534,617, U.S. Pat. No. 6,143,523, U.S. Pat. No. 6,022,711, U.S. Pat. Nos. 5,834,598, and 5,688,666. The term “substantially has the following amino acid sequence” means that the above amino acid sequence may include one or more amino-acid changes (deletion, addition, insertion or replacement) as long as such changes will not cause any disadvantageous non-similarity in function to human growth hormone receptor antagonists. It is more preferable to use the human growth hormone receptor antagonists substantially having an amino acid sequence, in which at least one lysine, aspartic acid, glutamic acid, unpaired cysteine residue, a free N-terminal α-amino group or a free C-terminal carboxyl group, is included.

The term “hGH receptor antagonist”, when used herein, encompasses all human Growth Hormone receptor antagonists, as well as their variants, derivatives, and fragments thereof that are characterized by being antagonists of the hGH receptor. Illustrating but not limiting examples of amino acid sequences of such hGH receptor antagonist are discussed below and in sequence databases such as Genseq, Swissprot, Genbank, Embl, and PIR.

Preferably, the term “hGH receptor antagonist” refers to the hGH receptor antagonist of SEQ ID NO:1 as well as its variants, homologs and derivatives exhibiting essentially the same biological activity. More preferably, the term “hGH receptor antagonist” refers to the polypeptide of SEQ ID NO 1.

The term “hGH receptor antagonist variants”, as used herein, refers to polypeptides from the same species but differing from a reference hGH receptor antagonist. Generally, differences are limited so that the amino acid sequences of the reference and the variant are closely similar overall and, in many regions, identical. Preferably, hGH receptor antagonists are at least 70%, 80%, 90%, 95%, 96%, 97%, 98% or 99% identical to a reference hGH receptor antagonist, preferably the hGH receptor antagonist of SEQ ID NO:1. By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence. The query sequence may be an entire amino acid sequence of the reference sequence or any fragment specified as described herein.

It is known in the art that one or more amino acids may be deleted from the N-terminus or C-terminus of a bioactive peptide or protein without substantial loss of biological function (see for instance, Ron et al., (1993), Biol. Chem., 268 2984-2988; which disclosure is hereby incorporated by reference in its entirety).

It also will be recognized by one of ordinary skill in the art that some amino acid sequences of hGH receptor antagonists can be varied without significant effect of the structure or function of the protein. Such mutants include deletions, insertions, inversions, repeats, and substitutions selected according to general rules known in the art so as to have little effect on activity. For example, guidance concerning how to make phenotypically silent amino acid substitutions is provided in Bowie et al. (1990), Science 247:1306-1310, hereby incorporated by reference in its entirety, wherein the authors indicate that there are two main approaches for studying the tolerance of an amino acid sequence to change.

Typically seen as conservative substitutions are the replacements, one for another, among the aliphatic amino acids Ala, Val, Leu and Phe; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. In addition, the following groups of amino acids generally represent equivalent changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; (5) Phe, Tyr, Trp, His.

As used herein, the term “hGH receptor antagonist fragment” refers to any peptide or polypeptide comprising a contiguous span of a part of the amino acid sequence of an hGH receptor antagonist, preferably the polypeptide of SEQ ID NO:1.

More specifically, a hGH receptor antagonist fragment comprising at least 6, preferably at least 8 to 10, more preferably 12, 15, 20, 25, 30, 35, 40, 50, 60, 75, 100, 125, 150, 175 or 191 consecutive amino acids of a hGH receptor antagonist according to the present invention. hGH receptor antagonist fragment may additionally be described as sub-genera of hGH receptor antagonists comprising at least 6 amino acids, wherein “at least 6” is defined as any integer between 6 and the integer representing the C-terminal amino acid of a hGH receptor antagonist including the polypeptide of SEQ ID NO:1. Further included are species of hGH receptor antagonist fragments at least 6 amino acids in length, as described above, that are further specified in terms of their N-terminal and C-terminal positions. Also encompassed by the term “hGH receptor antagonist fragment” as individual species are all hGH receptor antagonist fragments, at least 6 amino acids in length, as described above, that may be particularly specified by a N-terminal and C-terminal position. That is, every combination of a N-terminal and C-terminal position that a fragment at least 6 contiguous amino acid residues in length could occupy, on any given amino acid sequence of the sequence listing or of the present invention is included in the present invention.

Also encompassed by the term “hGH receptor antagonist fragment” are domains of hGH receptor antagonists. Such domains may eventually comprise linear or structural motifs and signatures including, but not limited to, leucine zippers, helix-turn-helix motifs, post-translational modification sites such as glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites. Such domains may present a particular biological activity such as DNA or RNA-binding, secretion of proteins, transcription regulation, enzymatic activity, substrate binding activity, etc. . . .

A domain has a size generally comprised between 3 and 191 amino acids. In preferred embodiment, domains comprise a number of amino acids that is any integer between 6 and 191. Domains may be synthesized using any methods known to those skilled in the art, including those disclosed herein for the preparation of hGH receptor antagonists to produce antibodies. Methods for determining the amino acids that make up a domain with a particular biological activity include mutagenesis studies and assays to determine the biological activity to be tested.

The identity percentage is determined after optimal alignment of two polynucleotides or polypeptide sequences over a comparison window, wherein portions of the polynucleotide or polypeptide sequences in the comparison window may comprise additions or deletions of one or more residue in order to optimize sequence alignment. The comparison window contains a certain number of positions (either a residue or a gap corresponding to an insertion/deletion of a residue), this number of positions corresponding to the window size. Each window position may present one of the following situations:

1°/There is a residue (nucleotide or amino acid) on this position on the first aligned sequence and a different residue at the same position on the second aligned sequence, in other words the second sequence has a substituted residue at this position compared to the first sequence.

2°/There is a residue (nucleotide or amino acid) on this position on the first aligned sequence and the same residue at the same position on the second aligned sequence.

3°/There is a residue (nucleotide or amino acid) on this position on the first aligned sequence and no residue at the same position on the second aligned sequence, in other words the second sequence presents a deletion at this position compared to the first sequence.

The number of positions within the comparison window belonging to the first above-defined category is called R1.

The number of positions within the comparison window belonging to the second above-defined category is called R2.

The number of positions within the comparison window belonging to the third above-defined category is called R3.

The identity percentage (% id) is may be calculated by any of the following formulas:

% id=R2/(R1+R2+R3)×100, or

% id=(R2+R3)/(R1+R2+R3)×100

Alignment of sequences to compare may be performed using any of the variety of sequence comparison algorithms and programs known in the art. Such algorithms and programs include, but are by no means limited to, TBLASTN, BLASTP, FASTA, TFASTA, FASTDB, WU-BLAST, Gapped-BLAST, PSI-BLAST (Pearson and Lipman, (1988), Proc. Natl. Acad. Sci. USA 85:2444-2448; Altschul et al., (1990), J. Mol. Biol. 215:403-410; Altschul et al., (1993), Nature Genetics 3:266-272; Altschul et al., (1997), Nuc. Acids Res. 25:3389-3402; Thompson et al., (1994), Nuc. Acids Res. 22:4673-4680; Higgins et al., (1996), Meth. Enzymol. 266:383-402; Brutlag et al. (1990) Comp. App. Biosci. 6:237-245; Jones and Swindells, (2002) Trends Biochem Sci 27:161-4; Olsen et al. (1999) Pac Symp Biocomput; 302-13), the disclosures of which are incorporated by reference in their entireties.

In a particular embodiment, the Smith-Waterman method is used with scoring matrix such as PAM, PAM 250 or preferably with BLOSUM matrices such as BLOSUM60 or BLOSUM62 and with default parameters (Gap Opening Penalty=10 and Gap Extension Penalty=1) or with user-specified parameters preferably superior to default parameters.

In another particular embodiment, protein and nucleic acid sequences are aligned using the Basic Local Alignment Search Tool (“BLAST”) programs with the default parameters or with modified parameters provided by the user. Preferably, the scoring matrix used is the BLOSUM62 matrix (Gonnet et al., (1992), Science 256:1443-1445; Henikoff and Henikoff, (1993), Proteins 17:49-61, which disclosures are hereby incorporated by reference in their entireties). Less preferably, the PAM or PAM250 matrices may also be used (see, e.g., Schwartz and Dayhoff, (1978), eds., Matrices for Detecting Distance Relationships: Atlas of Protein Sequence and Structure, Washington: National Biomedical Research Foundation, which disclosure is hereby incorporated by reference in its entirety).

In still another particular embodiment, polynucleotide or polypeptide sequences are aligned using the FASTDB computer program based on the algorithm of Brutlag et al. (1990), supra. Preferred parameters used in a FASTDB alignment of DNA sequences are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the length of the subject nucleotide sequence, whichever is shorter. Preferred parameters used in a FASTDB amino acid alignment are: Matrix=PAM 0, k-tuple=2, Mismatch Penalty=1, Joining Penalty=20, Randomization Group25Length=0, Cutoff Score=1, Window Size=sequence length, Gap Penalty=5, Gap Size Penalty=0.05, Window Size=500 or the length of the subject amino acid sequence, whichever is shorter.

Exemplary human growth hormone receptor antagonists are human growth hormone variants having at least one amino acid substitution of the lysine at positions 41 and the leucine at position 45, and particularly isoleucine or arginine at position 41 and tryptophan at position 45 (U.S. Pat. No. 5,534,617). Further exemplary human growth hormone receptor antagonists are human growth hormone variants having at least two amino acid substitutions at positions 54, 56, 58, 64, and particularly 54P 56D, 58T 64K, 54P 56W 58T 64K, and 54P 64K (U.S. Pat. No. 5,534,617).

Additional exemplary human growth hormone receptor antagonists are human growth hormone variants having greater affinity for the growth hormone receptor at Site I (U.S. Pat. No. 6,022,711). Particular exemplary human growth hormone receptor antagonists are human growth hormone variants having the amino acid substitutions:
H18D, H21N, R167N, K168A, D171S, K172R, E174S, I179T;
H18D, Q22A, F25A, D26A, Q29A, E65A, K168A, E174S;
H18A, Q22A, F25A, D26A, Q29A, E65A, K168A, E174S;
H18D, Q22A, F25A, D26A, Q29A, E65A, K168A, E174A (U.S. Pat. No. 6,022,711).

Additional exemplary human growth hormone receptor antagonists are human growth hormone variants having amino acid substitutions at, positions 10, 14, 18, and 21. Particular exemplary human growth hormone receptor antagonists are human growth hormone variants having the amino acid substitutions 10H, 14G, 18N, 21N; 10A, 14W, 18D, 21N; 10Y, 14T, 18V, 21N; and 10I, 14N, 18I, 21N (U.S. Pat. No. 5,834,598). Further exemplary human growth hormone receptor antagonists are human growth hormone variants having the amino acid substitutions 174S and 176Y and one or more amino acid substitutions at positions 10, 14, 18, 21, 167, 171, 175, and 179. Further exemplary human growth hormone receptor antagonists are human growth hormone variants having eight naturally occurring hGH amino acids F10, M14, H18, H21, R167, D171, T175 and I179 respectively are as a group replaced with a corresponding amino acid sequentially selected from the group consisting of:
H, G, N, N, N, S, T, T;
H, G, N, N, E, S, T, I;
H, G, N, N, N, N, T, T;
A, W, D, N, N, S, T, T;
A, W, D, N, E, S, T, I;
A, W, D, N, N, T, T, T;
F, S, F, L, N, S, T, T;
F, S, F, L, E, S, T, I;
F, S, F, L, N, N, T, T.
H, G, N, N, N, S, T, N;
A, N, D, A, N, N, T, N;
F, S, F, G, H, S, T, T;
H, Q, T, S, A, D, N, S;
H, G, N, N, N, A, T, T;
F, S, F, L, S, D, T, T;
A, S, T, N, R, D, T, I;
Q, Y, N, N, H, S, T, T;
W, G, S, S, R, D, T, I;
F, L, S, S, K, N, T, V;
W, N, N, S, H, S, T, T;
A, N, A, S, N, S, T, T;
P, S, D, N, R, D, T, I;
H, G, N, N, N, N, T, S;
F, S, T, G, R, D, T, I;
M, T, S, N, Q, S, T, T;
F, S, F, L, T, S, T, S;
A, W, D, N, R, D, T, I;
A, W, D, N, H, S, T, N;
M, Q, M, N, N, S, T, T;
H, Y, D, H, R, D, T, T;
L, N, S, H, R, D, T, I;
L, N, S, H, T, S, T, T;
A, W, D, N, N, A, T, T;
F, S, T, G, R, D, T, I;
A, W, D, N, R, D, T, I;
I, Q, E, H, N, S, T, T;
F, S, L, A, N, S, T, V;
F, S, F, L, K, D, T, T;
M, A, D, N, N, S, T, T;
A, W, D, N, S, S, V, T; and
H, Q, Y, S, R, D, T, I (U.S. Pat. No. 5,834,598).

The substitution of a different amino acid at G120 is one modification that disrupts Site 2 binding. Accordingly, an hGH variant including an amino acid substitution at G120 acts as an hGH antagonist. The human growth hormone receptor antagonist could be modified at residue 120 from a glycine to any more bulky amino acid. Specific substitutions at residue 120 are lysine and cysteine. Specific embodiments are human growth hormone receptor antagonists wherein a G120 amino acid substitution is combined with sets of Site 1 amino acid substitutions (U.S. Pat. No. 5,849,535). Thus, in one embodiment, an human growth hormone receptor antagonist includes the following set of amino acid substitutions:

H18D, H21N, G120K, R167N, K168A, D171S, K172R, E174S, I179T (hereinafter the “B2036 variant”).

In another embodiment, the human growth hormone receptor antagonist includes the following set of amino acid substitutions:

H18A, Q22A, F25A, D26A, Q29A, E65A, G120K, K168A, E174A (hereinafter the “B2024 variant”).

According to the present invention, poly(ethylene glycol) is covalently bound through amino acid residues of human growth hormone receptor antagonists. The amino acid residue may be any reactive one(s) having, for example, free amino, carboxyl, sulfhydryl(thiol), hydroxyl, guanidinyl, or imidizoyl groups, to which a terminal reactive group of an activated poly(ethylene glycol) may be bound. The amino acid residues having the free amino groups may include lysine residues and/or N-terminal amino acid residue, those having a free carboxyl group may include aspartic acid, glutamic acid and/or C-terminal amino acid residues, those having a free sulfhydryl(thiol) such as cysteine, those having a free hydroxyl such as serine or tyrosine, those having a free guanidinyl such as arginine, and those having a free imidizoyl such as histidine.

In another embodiment, oxime chemistries (Lemieux & Bertozzi Tib Tech 16:506-513, 1998) are used to target N-terminal serine residues.

The poly(ethylene glycol) used in the present invention is not restricted to any particular form or molecular weight range. The poly(ethylene glycol) molecular weight may between about 500 and about 100,000 Dalton. The term “about” indicating that in preparations of polyethylene glycol, some molecules will weigh more, some less, than the stated molecular weight and the stated molecular weight refers to the average molecular weight. It is understood that there is some degree of polydispersity associated with polymers such as poly(ethylene glycol). It is preferable to use PEGs with low polydispersity. Normally, a PEG with molecular weight of about 500 to about 60,000 is used. A specific PEG molecular weight range of the present invention is from about 1,000 to about 40,000. In another specific embodiment the PEG molecular weight is greater than about 5,000 to about 40,000. In another specific embodiment the PEG molecular weight about 20,000 to about 40,000. Other sizes may be used, depending on the desired therapeutic profile (e.g. duration of sustained release desired, the effects, if any on biological activity, the degree or lack of antigenicity and other known effects of the polyethylene to a therapeutic protein. For example the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 45,000, 50,000, 55,000, 60,000, 65,000, 70,000, 75,000, 80,000, 85,000, 90,000, 95,000, or 100,000 Dalton. The poly(ethylene glycol) can also be a branched PEG as described in U.S. Pat. No. 5,932,462, U.S. Pat. No. 5,342,940, U.S. Pat. No. 5,643,575, U.S. Pat. No. 5,919,455, U.S. Pat. No. 6,113,906, and U.S. Pat. No. 5,183,660.

Poly(alkylene oxides), notably poly(ethylene glycol)s, are bound to human growth hormone receptor antagonists via a terminal reactive group, which may or may not leave a linking moiety (spacer) between the PEG and the protein. In order to form the human growth hormone receptor antagonist conjugates of the present invention, polymers such as poly(alkylene oxide) are converted into activated forms, as such term is known to those of ordinary skill in the art. The reactive group, for example, is a terminal reactive group, which mediates a bond between chemical moieties on the protein, such as amino, carboxyl or thiol groups, and poly(ethylene glycol). Typically, one or both of the terminal polymer hydroxyl end-groups, (i.e. the alpha and omega terminal hydroxyl groups) are converted into reactive functional groups, which allows covalent conjugation. This process is frequently referred to as “activation” and the poly(ethylene glycol) product having the reactive group is hereinafter referred to as “an activated poly(ethylene glycol)”. Polymers containing both α and ω linking groups are referred to as “bis-activated poly(alkylene oxides)” and are referred to as “bifunctional”. Polymers containing the same reactive group on α and ω terminal hydroxyls are sometimes referred to as “homobifunctional” or “homobis-activated”. Polymers containing different reactive groups on α and ω terminal hydroxyls are sometimes referred to as “heterobifunctional” or “heterobis-activated”. Polymers containing a single reactive group are referred to as “mono-activated” polyalkylene oxides or “mono-functional”. Other substantially non-antigenic polymers are similarly “activated” or “functionalized”.

The activated polymers are thus suitable for mediating a bond between chemical moieties on the protein, such as α- or ε-amino, carboxyl or thiol groups, and poly(ethylene glycol). Bis-activated polymers can react in this manner with two protein molecules or one protein molecule and a reactive small molecule in another embodiment to effectively form protein polymers or protein-small molecule conjugates through cross linkages. Functional groups capable of reacting with either the amino terminal α-amino group or ε-amino groups of lysines found on the human growth hormone receptor antagonists include: N-hydroxysuccinimidyl esters, carbonates such as the p-nitrophenyl, or succinimidyl; carbonyl imidazole; azlactones; cyclic imide thiones; isocyanates or isothiocyanates; tresyl chloride (EP 714 402, EP 439 508); and aldehydes. Functional groups capable of reacting with carboxylic acid groups, reactive carbonyl groups and oxidized carbohydrate moieties on human growth hormone receptor antagonists include; primary amines; and hydrazine and hydrazide functional groups such as the acyl hydrazides, carbazates, semicarbamates, thiocarbazates, etc. Mercapto groups, if available on the human growth hormone receptor antagonists, can also be used as attachment sites for suitably activated polymers with reactive groups such as thiols; maleimides, sulfones, and phenyl glyoxals; see, for example, U.S. Pat. No. 5,093,531, the disclosure of which is hereby incorporated by reference. Other nucleophiles capable of reacting with an electrophilic center include, but are not limited to, for example, hydroxyl, amino, carboxyl, thiol, active methylene and the like.

Also included are polymers including lipophilic and hydrophilic moieties disclosed in U.S. Pat. No. 5,359,030 and U.S. Pat. No. 5,681,811; U.S. Pat. No. 5,438,040; and U.S. Pat. No. 5,359,030.

In one preferred embodiment of the invention secondary amine or amide linkages are formed using the human growth hormone receptor antagonists N-terminal α-amino group or εamino groups of lysine and the activated PEG. In another preferred aspect of the invention, a secondary amine linkage is formed between the N-terminal primary α- or ε-amino group of human growth hormone receptor antagonists and single or branched chain PEG aldehyde by reduction with a suitable reducing agent such as NaCNBH₃, NaBH₃, Pyridine Borane etc. as described in Chamow et al., Bioconjugate Chem. 5: 133-140 (1994) and U.S. Pat. No 5,824,784.

In another preferred embodiment of the invention, polymers activated with amide-forming linkers such as succinimidyl esters, cyclic imide thiones, or the like are used to effect the linkage between the human growth hormone receptor antagonists and polymer, see for example, U.S. Pat. No. 5,349,001; U.S. Pat. No. 5,405,877; and Greenwald, et al., Crit. Rev. Ther. Drug Carrier Syst. 17:101-161, 2000, which are incorporated herein by reference. One preferred activated poly(ethylene glycol), which may be bound to the free amino groups of human growth hormone receptor antagonists includes single or branched chain N-hydroxysuccinylimide poly(ethylene glycol) may be prepared by activating succinic acid esters of poly(ethylene glycol) with N-hydroxysuccinylimide.

Other preferred embodiments of the invention include using other activated polymers to form covalent linkages of the polymer with the human growth hormone receptor antagonists via ε-amino or other groups. For example, isocyanate or isothiocyanate forms of terminally activated polymers can be used to form urea or thiourea-based linkages with the lysine amino groups.

In another preferred aspect of the invention, carbamate (urethane) linkages are formed with protein amino groups as described in U.S. Pat. Nos. 5,122,614, 5,324,844, and 5,612,640, which are hereby incorporated by reference. Examples include N-succinimidyl carbonate, para-nitrophenyl carbonate, and carbonyl imidazole activated polymers. In another preferred embodiment of this invention, a benzotriazole carbonate derivative of PEG is linked to amino groups on human growth hormone receptor antagonists.

Another aspect of the invention represents a prodrug or sustained release form of human growth hormone receptor antagonists, comprised of a water soluble polymer, such as poly(ethylene glycol), attached to an human growth hormone receptor antagonists molecule by a functional linker that can predictably break down by enzymatic or pH directed hydrolysis to release free human growth hormone receptor antagonists or other human growth hormone receptor antagonists derivative. The prodrug can also be a “double prodrug” (Bundgaard in Advanced Drug Delivery Reviews 3:39-65, 1989) involving the use of a cascade latentiation. In such systems, the hydrolytic reaction involves an initial rate-limiting (slow) enzymatic or pH directed step and a second step involving a rapid non-enzymatic hydrolysis that occurs only after the first has taken place. Such a releasable polymer provides protein conjugates, which are impermanent and could act as a reservoir, that continually discharge human growth hormone receptor antagonists. Such functional linkers are described in U.S. Pat. No. 5,614,549; U.S. Pat. No. 5,840,900; U.S. Pat. No. 5,880,131; U.S. Pat. No. 5,965,119; U.S. Pat. No. 6,011,042; U.S. Pat. No. 6,180,095 B1; Greenwald R. B. et al., J. Med. Chem. 42; 3657-3667, 1999; Lee, S. et al., Bioconjugate Chem 12:163-169, 2001; Garman A. J. et al., FEBS Lett. 223:361-365, 1987; Woghiren C. et al., Bioconjugate Chem. 4:314-318, 1993; Roberts M. J. et al., J. Pharm. Sci. 87; 1440-1445, 1998; Zhao X., in Ninth Int. Symp. Recent Adv. Drug Delivery Syst. 199; Greenwald R. B. et al., J. Med. Chem. 43:475-487, 2000; and Greenwald R. B. Crit. Rev. Ther. Drug Carrier Syst. 17:101-161, 2000.

The present invention relates to a method of using aldehyde chemistry to direct selectivity of the PEG moiety to the N-terminus using a butyrylaldehyde linker moiety.

An embodiment of the present invention is a human growth hormone receptor antagonist-PEG conjugate having the structure of Formula I or Formula II

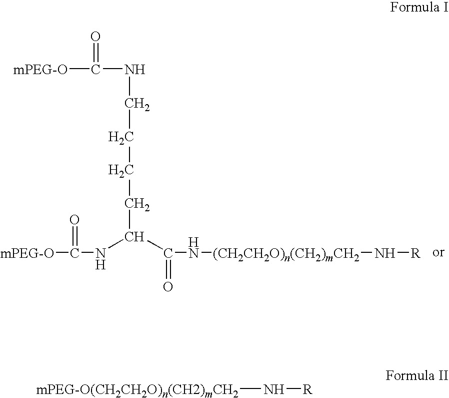

wherein

n is an integer between 1 and 10;

m is an integer between 1 and 10;

R is human growth hormone receptor antagonist.

In a particular embodiment n is between 1 and 5 and m is between 1 and 5.

In a particular embodiment of Formula I: n is 1 and m is 1; n is 1 and m is 2; n is 1 and m is 3; n is 1 and m is 4; n is 1 and m is 5; n is 1 and m is 6; n is 1 and m is 7; n is 1 and m is 8; n is 1 and m is 9; n is 1 and m is 10; n is 2 and m is 1; n is 2 and m is 2; n is 2 and m is 3; n is 2 and m is 4; n is 2 and m is 5; n is 2 and m is 6; n is 2 and m is 7; n is 2 and m is 8; n is 2 and m is 9; n is 2 and m is 10; n is 3 and m is 1; n is 3 and m is 2; n is 3 and m is 3; n is 3 and m is 4; n is 3 and m is 5; n is 3 and m is 6; n is 3 and m is 7; n is 3 and m is 8; n is 3 and m is 9; n is 3 and m is 10; n is 4 and m is 1; n is 4 and m is 2; n is 4 and m is 3; n is 4 and m is 4; n is 4 and m is 5; n is 4 and m is 6; n is 4 and m is 7; n is 4 and m is 8; n is 4 and m is 9; n is 4 and m is 10; n is 5 and m is 1; n is 5 and m is 2; n is 5 and m is 3; n is 5 and m is 4; n is 5 and m is 5; n is 5 and m is 6; n is 5 and m is 7; n is 5 and m is 8; n is 5 and m is 9; n is 5 and m is 10; n is 6 and m is 1; n is 6 and m is 2; n is 6 and m is 3; n is 6 and m is 4; n is 6 and m is 5; n is 6 and m is 6; n is 6 and m is 7; n is 6 and m is 8; n is 6 and m is 9; n is 7 and m is 10; n is 7 and m is 1; n is 7 and m is 2; n is 7 and m is 3; n is 7 and m is 4; n is 7 and m is 5; n is 7 and m is 6; n is 7 and m is 7; n is 7 and m is 8; n is 7 and m is 9; n is 7 and m is 10; n is 8 and m is 1; n is 8 and m is 2; n is 8 and m is 3; n is 8 and m is 4; n is 8 and m is 5; n is 8 and m is 6; n is 8 and m is 7; n is 8 and m is 8; n is 8 and m is 9; n is 8 and m is 10; n is 9 and m is 1; n is 9 and m is 2; n is 9 and m is 3; n is 9 and m is 4; n is 9 and m is 5; n is 9 and m is 6; n is 9 and m is 7; n is 9 and m is 8; n is 9 and m is 9; n is 9 and m is 10; n is 10 and m is 1; n is 10 and m is 2; n is 10 and m is 3; n is 10 and m is 4; n is 10 and m is 5; n is 10 and m is 6; n is 10 and m is 7; n is 10 and m is 8; n is 10 and m is 9; n is 10 and m is 10.

A specific embodiment is a human growth hormone receptor antagonist-PEG conjugate having the structure of the formula:

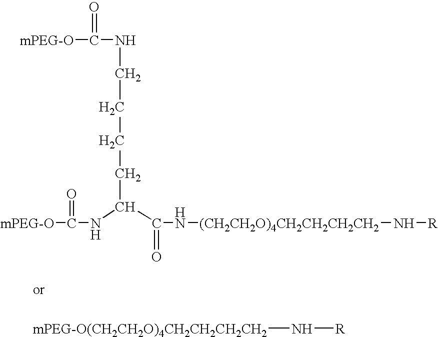

wherein R is human growth hormone receptor antagonist.

In a specific embodiment the human growth hormone receptor antagonists is the B2036 variant (SEQ ID NO:1).

A specific embodiment of the present invention is a human growth hormone-PEG conjugate wherein greater than 80%, more preferably 81%, more preferably 82%, more preferably 83%, more preferably 84%, more preferably 85%, more preferably 86%, more preferably 87%, more preferably 88%, more preferably 89%, more preferably 90%, more preferably 91%, more preferably 92%, more preferably 93%, more preferably 94%, more preferably 95%, more preferably 96%, more preferably 97, and more preferably 98% of the polyethylene glycol is conjugated to the amino-terminal phenylalanine of the amino acid sequence of SEQ ID NO:1.

Another specific embodiment of the present invention is a human growth hormone-PEG conjugate wherein greater than 90% of the polyethylene glycol is conjugated to the amino-terminal phenylalanine of the amino acid sequence of SEQ ID NO:1.

Another specific embodiment of the present invention is a human growth hormone-PEG conjugate wherein greater than 95% of the polyethylene glycol is conjugated to the amino-terminal phenylalanine of the amino acid sequence of SEQ ID NO:1.

Another specific embodiment of the present invention is a human growth hormone-PEG conjugate wherein greater than 98% of the polyethylene glycol is conjugated to an amino-terminal phenylalanine of the amino acid sequence of SEQ ID NO:1.

Conjugation reactions, referred to as pegylation reactions, were historically carried out in solution with molar excess of polymer and without regard to where the polymer will attach to the protein. Such general techniques, however, have typically been proven inadequate for conjugating bioactive proteins to non-antigenic polymers while retaining sufficient bioactivity. One way to maintain the human growth hormone receptor antagonist bioactivity is to substantially avoid the conjugation of those human growth hormone receptor antagonists reactive groups associated with the receptor binding site(s) in the polymer coupling process. Another aspect of the present invention is to provide a process of conjugating poly(ethylene glycol) to human growth hormone receptor antagonists maintaining high levels of retained activity.

The chemical modification through a covalent bond may be performed under any suitable condition generally adopted in a reaction of a biologically active substance with the activated poly(ethylene glycol). The conjugation reaction is carried out under relatively mild conditions to avoid inactivating the human growth hormone receptor antagonists. Mild conditions include maintaining the pH of the reaction solution in the range of 3 to 10 and the reaction temperatures within the range of from about 0°-37° C. In the cases where the reactive amino acid residues in human growth hormone receptor antagonists have free amino groups, the above modification is preferably carried out in a non-limiting list of suitable buffers (pH 3 to 10), including phosphate, MES, citrate, acetate, succinate or HEPES, for 1-48 hrs at 4°-37° C. In targeting N-terminal amino groups with reagents such as PEG aldehydes pH 4-7 is preferably maintained. The activated poly(ethylene glycol) may be used in about 0.05-100 times, preferably about 0.05-2.5 times, the molar amount of the number of free amino groups of human growth hormone receptor antagonists. On the other hand, where reactive amino acid residues in human growth hormone receptor antagonists have the free carboxyl groups, the above modification is preferably carried out in pH from about 3.5 to about 5.5, for example, the modification with poly(oxyethylenediamine) is carried out in the presence of carbodiimide (pH 4-5) for 1-24 hrs at 4°-37° C. The activated poly(ethylene glycol) may be used in 0.05-300 times the molar amount of the number of free carboxyl groups of human growth hormone receptor antagonists.

In separate embodiments, the upper limit for the amount of polymer included in the conjugation reactions exceeds about 1:1 to the extent that it is possible to react the activated polymer and human growth hormone receptor antagonists without forming a substantial amount of high molecular weight species, i.e. more than about 20% of the conjugates containing more than about one strand of polymer per molecule of human growth hormone receptor antagonists. For example, it is contemplated in this aspect of the invention that ratios of up to about 6:1 can be employed to form significant amounts of the desired conjugates which can thereafter be isolated from any high molecular weight species.

In another aspect of this invention, bifunctionally activated PEG derivatives may be used to generate polymeric human growth hormone receptor antagonist-PEG molecules in which multiple human growth hormone receptor antagonists molecules are crosslinked via PEG. Although the reaction conditions described herein can result in significant amounts of unmodified human growth hormone receptor antagonists, the unmodified human growth hormone receptor antagonists can be readily recycled into future batches for additional conjugation reactions. The processes of the present invention generate surprisingly very little, i.e. less than about 30% and more preferably, less than about 10%, of high molecular weight species and species containing more than one polymer strand per human growth hormone receptor antagonists. These reaction conditions are to be contrasted with those typically used for polymeric conjugation reactions wherein the activated polymer is present in several-fold molar excesses with respect to the target. In other aspects of the invention, the polymer is present in amounts of from about 0.1 to about 50 equivalents per equivalent of human growth hormone receptor antagonists. In other aspects of the invention, the polymer is present in amounts of from about 1 to about 10 equivalents per equivalent of human growth hormone receptor antagonists.

The conjugation reactions of the present invention initially provide a reaction mixture or pool containing mono- and di-PEG-human growth hormone receptor antagonist conjugates, unreacted human growth hormone receptor antagonist, unreacted polymer, and usually less than about 20% high molecular weight species. The high molecular weight species include conjugates containing more than one polymer strand and/or polymerized PEG-human growth hormone receptor antagonist species. After the unreacted species and high molecular weight species have been removed, compositions containing primarily mono- and di-polymer-human growth hormone receptor antagonist conjugates are recovered. Given the fact that the conjugates for the most part include a single polymer strand, the conjugates are substantially homogeneous. These modified human growth hormone receptor antagonists have at least about 0.1% of the in vitro biological activity associated with the native or unmodified human growth hormone receptor antagonists as measured using standard FDC-P1 cell proliferation assays, (Clark et al. Journal of Biological Chemistry 271:21969-21977, 1996), receptor binding assay (U.S. Pat. No. 5,057,417), or hypophysectomized rat growth (Clark et al. Journal of Biological Chemistry 271:21969-21977, 1996). In preferred aspects of the invention, however, the modified human growth hormone receptor antagonists have about 25% of the in vitro biological activity, more preferably, the modified human growth hormone receptor antagonists have about 50% of the in vitro biological activity, more preferably, the modified human growth hormone receptor antagonists have about 75% of the in vitro biological activity, and most preferably the modified human growth hormone receptor antagonists have equivalent or improved in vitro biological activity.

The processes of the present invention preferably include rather limited ratios of polymer to human growth hormone receptor antagonists. Thus, the human growth hormone receptor antagonist conjugates have been found to be predominantly limited to species containing only one strand of polymer. Furthermore, the attachment of the polymer to the human growth hormone receptor antagonists reactive groups is substantially less random than when higher molar excesses of polymer linker are used. The unmodified human growth hormone receptor antagonists present in the reaction pool, after the conjugation reaction has been quenched, can be recycled into future reactions using ion exchange or size exclusion chromatography or similar separation techniques.

A poly(ethylene glycol)-modified human growth hormone receptor antagonists, namely chemically modified protein according to the present invention, may be purified from a reaction mixture by conventional methods which are used for purification of proteins, such as dialysis, salting-out, ultrafiltration, ion-exchange chromatography, hydrophobic interaction chromatography (HIC), gel chromatography and electrophoresis. Ion-exchange chromatography is particularly effective in removing unreacted poly(ethylene glycol) and human growth hormone receptor antagonists. In a further embodiment of the invention, the mono- and di-polymer-human growth hormone receptor antagonist species are isolated from the reaction mixture to remove high molecular weight species, and unmodified human growth hormone receptor antagonists. Separation is effected by placing the mixed species in a buffer solution containing from about 0.5-10 mg/mL of the human growth hormone receptor antagonists-polymer conjugates. Suitable solutions have a pH from about 4 to about 10. The solutions preferably contain one or more buffer salts selected from KCl, NaCl, K₂HPO₄, KH₂PO₄, Na₂HPO₄, NaH₂PO₄, NaHCO₃, NaBO₄, CH₃CO₂H, and NaOH.

Depending upon the reaction buffer, the human growth hormone receptor antagonist polymer conjugate solution may first have to undergo buffer exchange/ultrafiltration to remove any unreacted polymer. For example, the PEG-human growth hormone receptor antagonists conjugate solution can be ultrafiltered across a low molecular weight cut-off (10,000 to 30,000 Dalton) membrane to remove most unwanted materials such as unreacted polymer, surfactants, if present, or the like.

The fractionation of the conjugates into a pool containing the desired species is preferably carried out using an ion exchange chromatography medium. Such media are capable of selectively binding PEG-human growth hormone receptor antagonist conjugates via differences in charge, which vary in a somewhat predictable fashion. For example, the number of available charged groups on the surface of the protein determines the surface charge of human growth hormone receptor antagonist. These charged groups typically serve as the point of potential attachment of poly(alkylene oxide) polymers. Therefore, human growth hormone receptor antagonist conjugates will have a different charge from the other species to allow selective isolation.

Strongly polar anion or cation exchange resins such as quaternary amine or sulfopropyl resins, respectively, are used for the method of the present invention. Ion exchange resins are especially preferred. A non-limiting list of included commercially available cation exchange resins suitable for use with the present invention are SP-hitrap®, SP Sepharose HP® and SP Sepharose® fast flow. Other suitable cation exchange resins e.g. S and CM resins can also be used. A non-limiting list of anion exchange resins, including commercially available anion exchange resins, suitable for use with the present invention are Q-hitrap®, Q Sepharose HP®, and Q Sepharose® fast flow. Other suitable anion exchange resins, e.g. DEAE resins, can also be used.

For example, the anion or cation exchange resin is preferably packed in a column and equilibrated by conventional means. A buffer having the same pH and osmolality as the polymer conjugated human growth hormone receptor antagonist solution is used. The elution buffer preferably contains one or more salts selected from KCl, NaCl, K₂HPO₄, KH₂PO₄, Na₂HPO₄, NaH₂PO₄, NaHCO₃, NaBO₄, and (NH₄)₂CO₃. The conjugate-containing solution is then adsorbed onto the column with unreacted polymer and some high molecular weight species not being retained. At the completion of the loading, a gradient flow of an elution buffer with increasing salt concentrations is applied to the column to elute the desired fraction of polyalkylene oxide-conjugated human growth hormone receptor antagonists. The eluted pooled fractions are preferably limited to uniform polymer conjugates after the cation or anion exchange separation step. Any unconjugated human growth hormone receptor antagonists species can then be back washed from the column by conventional techniques. If desired, mono and multiply pegylated human growth hormone receptor antagonist species can be further separated from each other via additional ion exchange chromatography or size exclusion chromatography.

Techniques utilizing multiple isocratic steps of increasing concentration can also be used. Multiple isocratic elution steps of increasing concentration will result in the sequential elution of di- and then mono-human growth hormone receptor antagonists-polymer conjugates.

The temperature range for elution is between about 4° C. and about 25° C. Preferably, elution is carried out at a temperature of from about 4° C. to about 22° C. For example, the elution of the PEG-human growth hormone receptor antagonist fraction is detected by UV absorbance at 280 nm. Fraction collection may be achieved through simple time elution profiles.

A surfactant can be used in the processes of conjugating the poly(ethylene glycol) polymer with the human growth hormone receptor antagonist moiety. Suitable surfactants include ionic-type agents such as sodium dodecyl sulfate (SDS). Other ionic surfactants such as lithium dodecyl sulfate, quaternary ammonium compounds, taurocholic acid, caprylic acid, decane sulfonic acid, etc. can also be used. Non-ionic surfactants can also be used. For example, materials such as poly(oxyethylene) sorbitans (Tweens), poly(oxyethylene) ethers (Tritons) can be used. See also Neugebauer, A Guide to the Properties and Uses of Detergents in Biology and Biochemistry (1992) Calbiochem Corp. The only limitations on the surfactants used in the processes of the invention are that they are used under conditions and at concentrations that do not cause substantial irreversible denaturation of the human growth hormone receptor antagonists and do not completely inhibit polymer conjugation. The surfactants are present in the reaction mixtures in amounts from about 0.01-0.5%; preferably from 0.05-0.5%; and most preferably from about 0.075-0.25%. Mixtures of the surfactants are also contemplated.

It is thought that the surfactants provide a temporary, reversible protecting system during the polymer conjugation process. Surfactants have been shown to be effective in selectively discouraging polymer conjugation while allowing lysine-based or amino terminal-based conjugation to proceed.

The present poly(ethylene glycol)-modified human growth hormone receptor antagonists have a more enduring pharmacological effect, which may be possibly attributed to its prolonged half-life in vivo.

The chemically modified human growth hormone receptor antagonists of the present invention, are useful in treating conditions in which the inhibition of GH action is desirable. Particularly amenable to treatment with chemically modified human growth hormone receptor antagonists are conditions in which a reduction of circulating levels of GH or of a mediator of GH action, such as IGF-I, provides a therapeutic benefit. Such conditions include conditions of GH excess such as, for example, giantism and acromegaly. Giantism results from GH excess before puberty, when the long bone growth is still possible. Acromegaly results from GH excess after puberty, when the long bones have fused. Acromegaly is characterized by bony overgrowth and soft tissue swelling as well as hypertrophy of internal organs, especially the heart. Acromegaly is typically caused by a pituitary tumor that secretes GH. The hallmarks of the disease are high levels of circulating GH and IGF-I. The chemically modified human growth hormone receptor antagonists of the present invention are presently believed to offer a significant therapeutic benefit by inhibiting GH action.

The chemically modified human growth hormone receptor antagonists are also useful in treating the other conditions in which the inhibition of GH action provides therapeutic benefit. Examples include diabetes and its complications, such as for instance diabetic retinopathy and diabetic nephropathy. Diabetic retinopathy is characterized by proliferation of the cells making up the retinal blood vessels, growth of new vessels on top of the retina (neovascularization), development of microaneurysms, and leakage of fluid into the surrounding retinal tissue. The early hallmarks of diabetic nephropathy are renal hypertrophy and hyperfiltration. As the disease progresses, diffuse enlargement of the mesangial cells (which support the filtration apparatus of the kidney) is observed, accompanied by an absolute increase in the number of mesangial cells.

Vascular eye diseases that, like diabetic retinopathy, involve proliferative neovascularization are also believed to be amenable to treatment with antagonist human growth hormone receptor antagonist. Examples include retinopathy of prematurity, retinopathy associated with sickle cell anemia, and age-related macular degeneration, which is the most common cause of vision loss in persons over 55.

Other conditions in which the reduction of GH levels is presently believed to provide a therapeutic benefit include malignancies that respond to GH, or a mediator of GH action (such as IGF-1), by growing (hereinafter “GH-responsive malignancies”). Examples of GH-responsive malignancies include Wilm's tumor, various sarcomas (e.g., osteogenic sarcoma), and breast, colon, prostate, and thyroid cancer.

The chemically modified human growth hormone receptor antagonists of the present invention inhibit the growth of cells expressing receptors to which the variants bind. A wide variety of tissues express such receptors. For example, GH receptor mRNA is expressed in cell lines from normal placenta, thymus, brain, salivary gland, prostate, bone marrow, skeletal muscle, trachea, spinal cord, retina, lymph node and from Burkitt's lymphoma, colorectal carcinoma, lung carcinoma, lymphoblastic leukemia, and melanoma. Thus, it is presently believed that chemically modified human growth hormone receptor antagonists of the present invention are generally useful in treating cancers that express receptors to which the variants bind.

The present poly(ethylene glycol)-modified human growth hormone receptor antagonists may be formulated into pharmaceuticals containing also a pharmaceutically acceptable diluent, an agent for preparing an isotonic solution, a pH-conditioner and the like in order to administer them into a patient.

The above pharmaceuticals may be administered subcutaneously, intramuscularly, intravenously, pulmonary, intradermally, or orally, depending on a purpose of treatment. A dose may be also based on the kind and condition of the disorder of a patient to be treated, being normally between 0.1 mg and 5 mg by injection and between 0.1 mg and 50 mg in an oral administration for an adult

The polymeric substances included are also preferably water-soluble at room temperature. A non-limiting list of such polymers include poly(alkylene oxide) homopolymers such as poly(ethylene glycol) or poly(propylene glycols), poly(oxyethylenated polyols), copolymers thereof and block copolymers thereof, provided that the water solubility of the block copolymers is maintained.

As an alternative to PEG-based polymers, effectively non-antigenic materials such as dextran, poly(vinyl pyrrolidones), poly(acrylamides), poly(vinyl alcohols), carbohydrate-based polymers, and the like can be used. Indeed, the activation of α- and ω-terminal groups of these polymeric substances can be effected in fashions similar to that used to convert poly(alkylene oxides) and thus will be apparent to those of ordinary skill. Those of ordinary skill in the art will realize that the foregoing list is merely illustrative and that all polymer materials having the qualities described herein are contemplated. For purposes of the present invention, “effectively non-antigenic” means all materials understood in the art as being nontoxic and not eliciting an appreciable immunogenic response in mammals.

### DEFINITIONS

The following is a list of abbreviations and the corresponding meanings as used interchangeably herein:
g gram(s)
mg milligram(s)
ml or mL milliliter(s)
RT room temperature
PEG poly(ethylene glycol)

The complete content of all publications, patents, and patent applications cited in this disclosure are herein incorporated by reference as if each individual publication, patent, or patent application were specifically and individually indicated to be incorporated by reference.

Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

In the following examples, the human growth hormone receptor antagonist is that of SEQ ID NO:1. It is understood that other members of the human growth hormone receptor antagonist family of polypeptides could also be pegylated in a similar manner as exemplified in the subsequent examples.

All references, patents or applications cited herein are incorporated by reference in their entirety as if written herein.

The present invention will be further illustrated by referring to the following examples, which however, are not to be construed as limiting the scope of the present invention.

### EXAMPLES

### Example 1

### Branched Chain 40,000 MW PEG-ALD Human Growth Hormone Receptor Antagonists

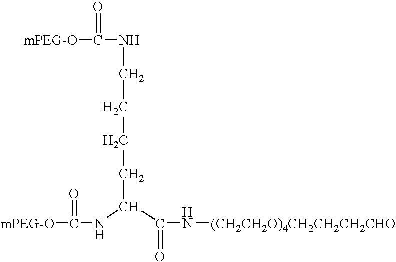

This example demonstrates a method for generation of substantially homogeneous preparations of N-terminally monopegylated human growth hormone receptor antagonist by reductive alkylation.

Methoxy-branched 40,000 MW PEG-aldehyde (PEG2 ALD) reagent (Shearwater Corp.) was selectively coupled via reductive amination to the N-terminus of human growth hormone receptor antagonist by taking advantage of the difference in the relative pKₐ value of the primary amine at the N-terminus versus pKₐ values of primary amines at the ε-amino position of lysine residues. Human growth hormone receptor antagonist protein dissolved at 10 mg/mL in 25 mM HEPES (Sigma Chemical St. Louis, Mo.) pH 7.1 was reacted with Methoxy-branched 40,000 MW PEG-aldehyde (PEG2-ALD) by addition of Methoxy-branched 40,000 MW PEG-aldehyde to yield a relative PEG:human growth hormone receptor antagonist molar ratio of 4:1. Reactions were catalyzed by addition of stock 1M NaCNBH₄ (Sigma Chemical, St. Louis, Mo.), dissolved in H₂0, or Pyiridine Borane complex to a final concentration of 10-50 mM. Reactions were carried out at 25° C. for 18-48 hours.

### Example 2

### Methoxy 20,000 MW PEG Aldehyde

Methoxy 20,000 MW PEG aldehyde (Shearwater) was coupled to human growth hormone receptor antagonist using the procedure described for Example 1.

### Example 3

### Methoxy 30,000 MW PEG Aldehyde

Methoxy 30,000 MW PEG aldehyde (Shearwater) was coupled to human growth hormone receptor antagonist using the procedure described for Example 1.

### Example 4

### Methoxy-Branched 40,000 MW PEG-Butyraldehyde (PEG2-But ALD

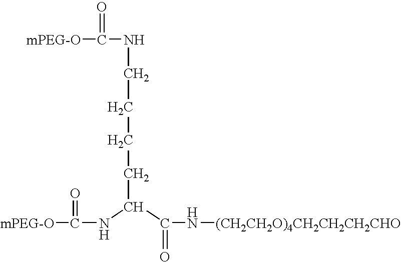

Methoxy-branched 40,000 MW PEG-Butyraldehyde (PEG2-But ALD) reagent (Shearwater Corp.) is coupled to the N-terminus of human growth hormone receptor antagonist using the procedure described for Example 1.

### Example 5

### Branched 40,000 MW PEG2 NHS-PEG-Human Growth Hormone Receptor Antagonist

40,000 MW branched PEG2-NHS (Shearwater Corp.) was coupled to human growth hormone receptor antagonist using the procedure described for Example 1.

### Example 6

### Purification of Pegylated hGH Receptor Antagonists

Pegylated hGH receptor antagonist species were purified from the reaction mixture to >95% (SEC analysis) using a single ion exchange chromatography step. As an example, Methoxy-branched 40,000 MW PEG-aldehyde was coupled to B2036. Reactions were carried out at 25 degrees C. for 60 min in 25 mM HEPES, pH 7.1 at a protein concentration of 10 mg/mL using a PEG:Protein molar ratio of 4:1. Mono PEGylated human growth hormone receptor antagonist was purified from the reaction mixture using a Q Sepharose HP column equilibrated in 25 mM HEPES buffer pH 7.3 and a linear NaCl gradient.

### Anion Exchange Chromatography

Mono-pegylated 30K PEG-aldehyde, 20K PEG aldehyde, 40K PEG NHS, and branched 40K PEG aldehyde hGH receptor antagonist species were purified from the reaction mixture to >95% (SEC analysis) using a single anion exchange chromatography step. Mono-pegylated hGH receptor antagonist was purified from unmodified receptor antagonist and multi-pegylated hGH receptor antagonist species using anion exchange chromatography. A typical Methoxy-branched 40,000 MW PEG-aldehyde and hGH receptor antagonist reaction mixture (5-100 mg protein), as described above, was purified on a Q-Sepharose Hitrap column (1 or 5 mL) (Amersham Pharmacia Biotech, Piscataway, N.J.) or Q-Sepharose fast flow column (26/20, 70 mL bed volume) (Amersham Pharmacia Biotech, Piscataway, N.J.) equilibrated in 25 mM HEPES, pH 7.1 (Buffer A). The reaction mixture was diluted 5-10× with buffer A and loaded onto the column at a flow rate of 2.5 mL/min. The column was washed with 8 column volumes of buffer A. Subsequently, the various hGH species were eluted from the column in 80-100 column volumes of Buffer A and a linear NaCl gradient of 0-200 mM. The eluant was monitored by absorbance at 280 nm (A₂₈₀) and fractions were collected. Fractions were pooled as to extent of pegylation, e.g., mono, di. The pool was then concentrated to 0.5-5 mg/mL in a Centriprep YM10 concentrator (Amicon, Technology Corporation, Northborough, Mass.). Protein concentration of pool was determined by A₂₈₀ using an extinction coefficient of 0.78. Total yield of purified mono branched 40,000 MW PEG-aldehyde from this process was 44%.

### Cation Exchange Chromatography

Cation exchange chromatography is carried out on an SP Sepharose high performance column (Pharmacia XK 26/20, 70 ml bed volume) equilibrated in 10 mM sodium acetate pH 4.0 (Buffer B). The reaction mixture is diluted 10× with buffer B and loaded onto the column at a flow rate of 5 mL/min. Next the column is washed with 5 column volumes of buffer B, followed by 5 column volumes of 12% buffer C (10 mM acetate pH 4.5, 1 M NaCl). Subsequently, the PEG-hGH species are eluted from the column with a linear gradient of 12 to 27% buffer C in 20 column volumes. The eluant is monitored at 280 nm and 10 mL fractions are collected. Fractions are pooled according to extent of pegylation (mono, di, tri etc.), exchanged into 10 mM acetate pH 4.5 buffer and concentrated to 1-5 mg/mL in a stirred cell fitted with an Amicon YM10 membrane. Protein concentration of pool is determined by A280 nm using an extinction coefficient of 0.78.

### Example 7

### Biochemical Characterization

The purified pegylated hGH receptor antagonist conjugates were characterized by reducing and non-reducing SDS-PAGE, non-denaturing Size Exclusion Chromatography, analytical anion Exchange Chromatography, N-terminal Sequencing, Hydrophobic Interaction chromatography, and reversed phase HPLC.

### Size Exclusion High Performance Liquid Chromatography (SEC-HPLC)

### Non-Denaturing SEC-HPLC

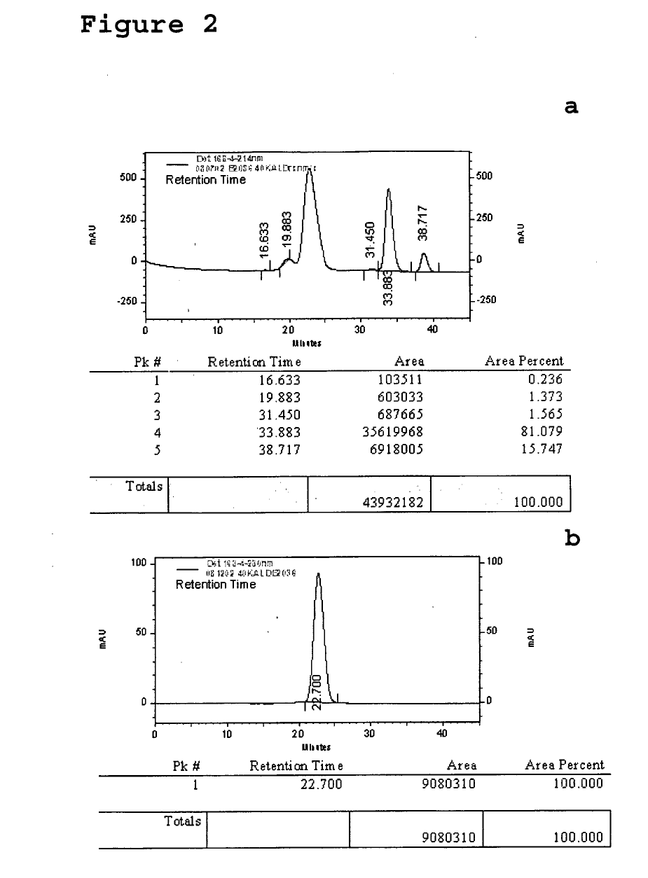

The reaction of various sizes and geometries with hGH receptor antagonists, anion exchange purification pools and final purified products were assessed using non-denaturing SEC-HPLC. Analytical non-denaturing SEC-HPLC was carried out using a Tosohaas G4000PWXL column, 7.8 mm×30 cm, (Tosohaas Pharmacia Biotech, Piscataway, N.J.) in 20 mM Phosphate pH 7.2, 150 mM NaCl at a flow rate of 0.5 mL/minute. PEGylation greatly increases the hydrodynamic volume of the protein resulting in a shift to an earlier retention time. New species were observed in the PEG Methoxy-branched 40,000 MW PEG-Butyraldehyde hGH receptor antagonist reaction mixtures along with unmodified hGH receptor antagonist. These PEGylated and non-PEGylated species were separated on Q-Sepharose chromatography, and the resultant purified mono branched 40,000 MW PEG-Butyraldehyde hGH receptor antagonist species were subsequently shown to elute as a single peak on non-denaturing SEC (>95% purity) (FIG. 2b). The Q-Sepharose chromatography step effectively removed free PEG, hGH receptor antagonist, and di-PEGylated hGH receptor antagonist species from the mono-Pegylated hGH receptor antagonists (FIG. 2a). Non-denaturing SEC-HPLC demonstrated that the effective size of the various PEGylated-hGH receptor antagonist was much greater than their respective theoretical molecular weights.

### SDS PAGE

SDS-PAGE was used to assess the reaction of branched 40,000 MW PEG-aldehyde with hGH receptor antagonist and the purified final products (data not shown). SDS-PAGE was carried out on 1 mm thick 10-20% Tris tricine gels (Invitrogen, Carlsbad, Calif.) under reducing and non-reducing conditions and stained using a Novex Colloidal Coomassie™ G-250 staining kit (Invitrogen, Carlsbad, Calif.). Purified mono branched PEG-aldehyde hGH species migrate as one major band on SDS-PAGE.

### Analytical Anion Exchange HPLC

The reaction of Methoxy-branched 40,000 MW PEG-aldehyde with hGH receptor antagonists, anion exchange purification fractions and final purified products were assessed using analytical anion exchange HPLC. Analytical anion exchange HPLC was carried out using a Tosohaas Q5PW or DEAE-PW anion exchange column, 7.5 mm×75 mm (Tosohaas Pharmacia Biotech, Piscataway, N.J.) in 50 mM Tris ph 8.6 at a flow rate of 1 mL/min. Samples were eluted with a linear gradient of 5-200 mM NaCl.

### N-terminal Sequence and Peptide Mapping

Automated Edman degradation chemistry was used to determine the NH2-terminal protein sequence. An Applied Biosystems Model 494 Procise sequencer (Perkin Elmer, Wellesley, Mass.) was employed for the degradation. The respective PTH-AA derivatives were identified by RP-HPLC analysis in an on-line fashion employing an Applied Biosystems Model 140C PTH analyzer fitted with a Perkin Elmer/Brownlee 2.1 mm i.d. PTH-C18 column. Branched 40,000 MW PEG-Butyraldehyde human growth hormone receptor antagonist bands transferred to PVDF membranes or solutions of purified 20K linear and Methoxy-branched 40,000 MW PEG-Butyraldehyde hGH receptor antagonist conjugate were sequenced.

What is claimed is:

1. An amino-terminal monoPEGylated human growth hormone receptor antagonist conjugate.

2. The amino-terminal monoPEGylated conjugate of claim 1 having the structure of the Formula 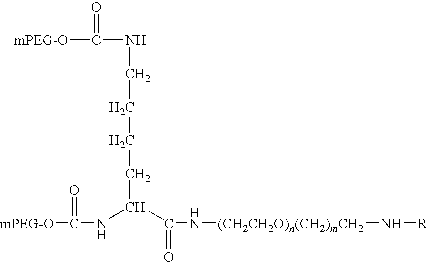
wherein
n is an integer between 1 and 10;
m is an integer between 1 and 10;
R is a human growth hormone receptor antagonist.

3. The PEG conjugate of claim 2 having the structure of the formula 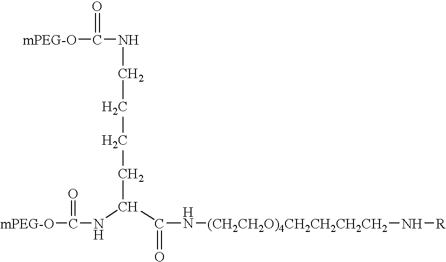
wherein R is a human growth hormone receptor antagonist.

4. The conjugate of claim 1, 2 or 3 wherein said human growth hormone receptor antagonist comprises an amino acid sequence of SEQ ID NO:1.

5. The conjugate of claim 1, 2 or 3 wherein said human growth hormone receptor antagonist consists of an amino acid sequence of SEQ ID NO:1.

6. The human growth hormone receptor antagonist-PEG conjugate of claim 4 wherein greater than 90% of said polyethylene glycol is conjugated to an amino-terminal phenylalanine of the amino acid sequence of SEQ ID NO:1.

7. The human growth hormone receptor antagonist-PEG conjugate of claim 4 wherein greater than 95% of said polyethylene glycol is conjugated to an amino-terminal phenylalanine of the amino acid sequence of SEQ ID NO:1.

8. A composition comprising the human growth hormone receptor antagonist-PEG conjugate of claim 1, 2, 3, 4, 5, 6 or 7, and at least one pharmaceutically acceptable carrier.

9. A method of treating a patient having a growth or development disorder comprising administering to said patient a therapeutically effective amount of the human growth hormone receptor antagonist-PEG conjugate of claim 1, 2, 3, 4, 5, 6 or 7.

10. The method of claim 9 wherein said growth or development disorder is giantism.

11. The method of claim 9 wherein said growth or development disorder is acromegaly.

12. The method of claim 9 wherein said growth or development disorder is diabetic retinopathy.

13. The method of claim 9 wherein said growth or development disorder is diabetic nephropathy.

14. A method of treating a patient having a GH-responsive malignancy comprising administering to said patient a therapeutically effective amount of the human growth hormone receptor antagonist-PEG conjugate of claim 1, 2, 3, 4, 5, 6 or 7.

15. A method of inhibit the growth of cells expressing receptors to which the variants bind comprising administering to a patient in need thereof a therapeutically effective amount of the human growth hormone receptor antagonist-PEG conjugate of claim 1, 2, 3, 4, 5, 6 or 7.


SEQUENCE LISTING (1 sequence)

SEQ ID NO: 1 - protein, 191 aa, Artificial Sequence
  human growth hormone antagonist B2036
FPTIPLSRLFDNAMLRADRLNQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEKIQTLMGRLEDGSPRTGQIFKQTYSKFDTNSHNDDALLKNYGLLYCFNADMSRVSTFLRTVQCRSVEGSCGF